(19) United States
(12) Patent Application Publication
(10) Pub. No.: US 2025/0215064 A1
(43) Pub. Date: Jul. 3, 2025

(54) BISPECIFIC T CELL ENGAGER AND USES THEREOF

(71) Applicant: MANYSMART THERAPEUTICS, INC., Tapei City (TW)

(72) Inventors: HSIN-YI HUANG, TAIPEI CITY (TW); CHENG HAO LIAO, TAIPEI CITY (TW); CHUN-MING LIN, TAIPEI CITY (TW)

(73) Assignee: MANYSMART THERAPEUTICS, INC., Tapei City (TW)

(21) Appl. No.: 19/031,539

(22) Filed: Jan. 18, 2025

Related U.S. Application Data
(62) Division of application No. 17/057,957, filed on Nov. 23, 2020, now Pat. No. 12,269,863.
(60) Provisional application No. 62/675,208, filed on May 23, 2018.

(51) Int. Cl. C07K 14/735; A61K 39/00; A61P 31/22; A61P 35/00; C07K 16/28

(52) U.S. Cl. CPC C07K 14/70535; A61P 31/22; A61P 35/00; C07K 16/2809; A61K 2039/505; C07K 2317/622; C07K 2317/732; C07K 2319/02

(57) ABSTRACT

A novel fusion protein to overcome the current difficulties related to application of monoclonal antibodies in disease treatment and in other fields, particularly those requiring ADCC, e.g. for depletion of tumor cells, virally-infected cells, or immune-modulating cells, etc. One example of the fusion protein is an extracellular domain of a high-affinity variant of human CD 16 A fused to an anti-CD3 antibody or its antigen-binding fragment thereof that specifically binds to an epitope on human CD3 or a fragment thereof.

### FIELD OF THE INVENTION

The present disclosure relates to a fusion protein, particularly, to a CD16A-bispecific T cell engager (BITE®) and uses thereof. This application is a DIV application of U.S. patent application Ser. No. 17/057,957, filed on 23 Nov. 2020, which is a national phase entry under 35 U.S.C. 371 of International Patent Application No.: PCT/US2019/033752 filed on 23 May 2019, which claims the benefit of U.S. Provisional Patent Application No. 62/675,208, filed on 23 May 2018, the entire content of which is incorporated by reference. The sequence listing filed in the U.S. patent application Ser. No. 17/057,957, filed on Nov. 23, 2020, is hereby also incorporated by reference in its entirety. An XML format of the sequence listing file titled “U-020661-DIV 19031539-US10873-Sequence Listing” (file size: 15 KB), created on Mar. 19, 2025, is filed as a separate part of the disclosure. The material in the XML file is hereby also incorporated by reference in its entirety.

### BACKGROUND OF THE INVENTION

Therapeutic monoclonal antibodies (mAbs) have become one of the fastest growing classes of drugs in recent years and are approved for the treatment of a wide range of indications, from cancer, infectious disease, and to autoimmune disease (Almagro et al., Front Immunol 8, 1751 (2017)). The majority of mAbs approved for use in oncology are the so-called direct-targeting mAbs, such as rituximab (an anti-CD20 mAb), which are designed to target tumor cells directly. This type of mAb is usually made ex vivo and passively injected into patients, where they act against established or residual tumors and thereby activate various Fc-receptor-mediated effector pathways to kill the target cells. When combined with chemotherapy, these therapeutic mAbs have achieved impressive results for hematologic malignancies, with anti-CD20 and anti-CD38 as examples of clinical efficacy in follicular lymphoma (Subramanian et al., Cancer Management and Research, 9, 131-140 (2017)) and multiple myeloma (van de Donk et al., Blood, 131, 13-29 (2018)), respectively. Instead, approved immunomodulatory mAbs, such as anti-CTLA-4 (Cytotoxic T-lymphocyte antigen 4), anti-PD-1 (Programmed Cell Death-1), and anti-PD-L1 (Programmed Death-Ligand1) are designed to block immune checkpoint to reactivate anti-tumor immune responses (K. Chin et al., Annals of Oncology, 28,1658-1666 (2017)), but may also function as direct-targeting mAbs to delete cells (Hamilton and Rath Expert Opinion on Biological Therapy, 17, 515-523 (2017)).

The mechanism of action of direct-targeting therapeutic mAbs stems from various natural functions of antibodies: neutralization, antibody-dependent cell-mediated cytotoxicity (ADCC), or complement-dependent cytotoxicity (Suzuki et al., J Toxicol Pathol, 28, 133-139 (2015)). The extent to which each mode of action contributes to clinical efficacy is unclear.

Therefore, there is need for developing a novel approach to improve therapeutic efficacy of mAbs and to boost mAb application in various fields. The present disclosure addressed these and other needs.

### SUMMARY OF THE INVENTION

The present disclosure provides a novel bispecific T cell engager to overcome the current difficulties related to application of mAbs in disease treatment and in other fields, particularly those requiring ADCC, e.g. depletion of tumor cells, virally-infected cells, or immune-modulating cells, etc.

The present disclosure provides a fusion protein comprising:
an extracellular domain of human CD16A; and
an antibody or antigen-binding fragment thereof that specifically binds to an epitope
on human CD3 or a fragment thereof.

The present disclosure also provides a polynucleotide encoding the fusion protein as disclosed herein.

The present disclosure also provides a host cell comprising the polynucleotide as disclosed herein.

The present disclosure also provides a pharmaceutical composition comprising a therapeutically effective amount of the fusion protein as disclosed herein and optionally a pharmaceutically acceptable carrier or excipient.

The present disclosure also provides use of the pharmaceutical composition as disclosed herein in the manufacture of a medicament for inducing antibody-dependent cellular cytotoxicity in a subject in need.

The present disclosure is described in detail in the following sections. Other characteristics, purposes and advantages of the present disclosure can be found in the detailed description and claims.

### BRIEF DESCRIPTION OF THE DRAWINGS

FIGS. 1A to 1G show genetic construction of the haCD16A-BiTE. FIG. 1A: The genetic construct of the haCD16A-CD3 bispecific T cell engager cloned in an adeno-associated virus (AAV) shuttle plasmid. H: 6×Histidine tag; ITR: inverted terminal repeat sequence of AAV; S: secretion signal; WPRE: woodchuck hepatitis B virus post-transcriptional regulatory element. FIG. 1B: The coding sequences of the extracellular domain (edCD16) of human high-affinity CD16A (haCD16A) and a single-chain antibody against human CD3 (anti-CD3 scFv) were fused in the same coding frame by gene synthesis. FIG. 1C: The synthesized gene with 1341 bp nucleotides encodes a fusion protein with 446 amino acids. FIG. 1D to 1G: Scheme of the haCD16A-BiTE.

FIGS. 2A to 2E show the binding of the haCD16A-BiTEs to T cells and immunoglobulin G (IgG) antibody-coated tumor cells. FIG. 2A: Analytical strategy of the haCD16A-BiTE binding to IgG antibody-coated cells. FIG. 2B: Antigen expression on tumor cells. FIG. 2C: The binding of the haCD16A-BiTE to IgG antibody-coated tumor cells. FIG. 2D: Analytical strategy of the haCD16A-BiTE binding to T cells. FIG. 2E: The binding of the haCD16A-BiTE to T cells.

FIG. 3 shows the effect on killing of CD20-expressing cell lines by treatment of said cell lines with anti-CD20 antibodies (Rituximab, Rituxin®) and the haCD16A-BiTEs alone or in combination in the presence of T cells.

FIG. 4 shows the effect on killing of epidermal growth factor receptor (EGFR)-expressing cell line by treatment of said cell line with anti-EGFR antibodies (Cetuximab, Erbitux®) and the haCD16A-BiTEs alone or in combination in the presence of T cells.

FIG. 5 shows the effect on killing of human epidermal growth factor receptor 2 (HER2)-expressing cell line by treatment of said cell line with anti-HER2 antibodies (Trastuzumab, Herceptin®) and the haCD16A-BiTEs alone or in combination in the presence of T cells.

FIG. 6 shows the effect of plasma on killing of CD20-expressing cell line treated with Rituximab and the haCD16A-BiTEs.

FIGS. 7A and 7B show comparison study of IgG antibody-mediated cell killing between CD16⁻γ9δ2 T cells pulsed with the haCD16A-BiTEs and CD16⁺γ9δ2 T cells.

FIG. 8 shows the effect on depletion of malignant B cells from T cell expansion culture by treatment of said cell expansion culture with anti-CD20 antibodies (Rituximab) and the haCD16A-BiTEs.

FIG. 9 shows the effect on killing of EBV-infected cell lines by treatment of said cell lines with anti-latent membrane protein 1 (LMP1) antibodies and the haCD16A-BiTEs alone or in combination in the presence of T cells.

FIG. 10 shows the effect on killing of PD-L1-expressing cell line by treatment of said cell line with anti-PD-L1 antibodies and the haCD16A-BiTEs alone or in combination in the presence of T cells.

FIG. 11 shows, in the presence of T cells, the in vivo effect of the combination therapy with the haCD16A-BiTEs and rituximab, and rituximab monotherapy on reducing cancer cell growth.

FIG. 12 shows the in vivo production of the haCD16A-BiTEs after AAV-mediated haCD16A-BiTE gene transfer.

### DETAILED DESCRIPTION OF THE INVENTION

The present disclosure provides a fusion protein comprising:
an Fc gamma receptor or ligand-binding fragment thereof; and
   an antibody or antigen-binding fragment thereof that specifically binds to an epitope on a surface antigen of T cells or a fragment thereof, wherein the surface antigen is able to trigger antibody-dependent cell-mediated cytotoxicity and/or to activate the T cells.

Particularly, the fusion protein according to the instant disclosure is known as a bispecific T cell engager that is provided with bispecific affinity to two antigens/ligands, acting as a bridge between a target cell and a T cell.

Preferably, the fusion protein activates ADCC immune response through which T cells can recognize and kill antibody-coated target cells expressing tumor-or pathogen-derived antigens on their surface.

Without combination with other treatment modalities, such as chemotherapy, monotherapies using direct-targeting of therapeutic mAbs to treat cancer generally result in limited therapeutic effects (Hiddemann et al., Blood, 106, 3725-32 (2005); Sehn et al., J Clin Oncol, 33, 3467-3474 (2015)). While not wishing to be limited by theory, the instant invention as disclosed herein is based on Applicant's idea that amplifying ADCC is a promising approach to increase the clinical benefits of therapeutic antibodies. The main immune effector cells mediating ADCC are natural killer (NK) cells (Wang et al., Front Immunol, 6, 368 (2015)). NK cells express Fc gamma receptors (FcγRs), mostly CD16A (FcγRIIIA), which recognize and bind to the Fc portion of IgG antibodies. Once the Fcγ receptor binds to the Fc region of IgG bound to the surface of target cells, the natural killer cell releases cytotoxic factors that cause the death of the target cell (Wang et al., Front Immunol, 6, 368 (2015)). Hence, ADCC involves three components: immune effector cells, antibodies, and target cells opsonized by the antibody. ADCC is triggered upon binding of FcγR expressed on the surface of NK cells to the Fc region of IgG molecules. It follows that affinity/quantities of FcγR and quality/quantities of NK cells could contribute to the differences in the magnitude of ADCC, given that genetic variation in FcγR resulting in different binding affinity is known to contribute to the differences in the magnitude of ADCC.

Examples of the Fc gamma receptors include but are not limited to CD16A, CD16B, CD32A, CD32B, CD64A, CD64B, and CD64C.

Examples of the antibody or antigen-binding fragment thereof that specifically binds to an epitope on a surface antigen of T cells or a fragment thereof include but are not limited to an anti-CD3 antibody, anti-4-1BB antibody, anti-CD28 antibody, or anti-OX40 antibody.

Preferably, the present disclosure provides a fusion protein comprising:
an extracellular domain of human CD16A; and
an antibody or antigen-binding fragment thereof that specifically binds to an epitope on human CD3 or a fragment thereof.

According to the disclosure, the fusion protein comprises an extracellular domain of CD16A. Preferably, the CD16A is human CD16A. CD16A, also known as FcγRIIIA, it is a transmembrane glycoprotein and there are two allelic variants of CD16A that have either a phenylalanine (F) or valine (V) residue at position 158. The CD16A-158V variant has a higher affinity for IgG, but CD16A-158F is the dominant allele in human population. Clinical analyses have revealed a positive correlation between the therapeutic efficacy of tumor-targeting therapeutic mAbs and CD16A binding affinity. Patients homozygous for the CD16A valine variant (CD16A-V/V) had an improved clinical outcome after treatment with anti-tumor therapeutic antibodies compared to those who were either heterozygous (CD16A-V/F) or homozygous (CD16A-F/F) for the lower affinity CD16A isoform in response to clinically approved therapeutic antibodies such as rituximab, trastuzumab, and cetuximab (Cartron et al., Blood, 99, 754-758 (2002); Kim et al., Blood, 108, 2720-2725 (2006); Zhang et al, J Clin Oncol, 25, 3712-3718 (2007); Musolino et al., J Clin Oncol, 26,1789-1796 (2008); Veeramani et al., Blood, 118, 3347-3349 (2011); Mellor et al., J Hematol Oncol, 6, 1 (2013)). However, only 10-20% of general population have high-affinity CD16A variant. In one preferred embodiment of the disclosure, the CD16A is a high-affinity CD16A variant.

In one more preferred embodiment of the disclosure, the extracellular domain of CD16A has an amino acid sequence of SEQ ID NO: 2 or a substantially similar sequence thereof.

As applied to polypeptides, the term “substantial similarity” or “substantially similar” means that a protein sequences, when optimally aligned with another (reference) protein sequence, such as by the programs GAP or BESTFIT using default gap weights, there is sequence identity in at least 90%, at least 95%, even more preferably at least 96%, 97%, 98% or 99% of amino acid residues, to the entire sequence of said reference protein sequence. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutant thereof. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

According to the disclosure, the fusion protein comprises an antibody or antigen-binding fragment thereof that specifically binds to an epitope on human CD3 or a fragment thereof.

In immune reponse, CD3 is a surface antigen associated with the T-cell receptor (TCR) to form a complex involved in antigen recognition and signal transduction. The CD3 T cell co-receptor helps to activate both the cytotoxic T cell (CD8⁺ T cells) and also T helper cells (CD4⁺ T cells). Utilizing the fusion protein according to the disclosure, all of the CD3-expressing cells, including alpha-beta T cells, gamma-delta T cells, and natural killer T cells in the body could potentially be recruited, through the anti-CD3 portion of the fusion protein binding to the CD3 molecules on T cells, to become cells carrying high-affinity CD16A and capable of carrying out ADCC.

Various techniques known to persons of ordinary skill in the art can be used to determine whether an antibody “specifically binds to one or more amino acids” within a polypeptide or protein. Exemplary techniques include, e.g., routine cross-blocking assay such as that described in Antibodies, by Harlow and Lane (Cold Spring Harbor Press, Cold Spring Harb., N.Y.), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can also be employed (Tomer, 2000, Protein Science 9:487-496). Another method that can be used to identify the amino acids within a polypeptide with which an antibody specifically binds is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues (bound by and) protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267 (2): 252-259; Engen and Smith (2001) Anal. Chem. 73: 256A-265A.

The antibody according to the disclosure can be full-length or may comprise only an antigen-binding portion, and may be modified to affect functionality as needed.

The term “antibody”, as used herein, means any antigen-binding molecule or molecular complex comprising at least one complementarity determining region (CDR) that specifically binds to or interacts with a particular antigen (e.g., CD3). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H1}, C_{H2} and C_{H3}. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-CD3 antibody (or antigen-binding portion thereof) may be identical to the human germline sequences, or may be naturally or artificially modified. An amino acid consensus sequence may be defined based on a side-by-side analysis of two or more CDRs.

The term “antibody”, as used herein, also includes an antigen-binding fragment of a full antibody molecule. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. An antigen-binding fragment of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of an antigen-binding fragment of an antibody includes: (i) Fab fragments; (ii) F (ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody typically comprises at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} Or V_{L}-V_{L} dimers.

Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H1}; (ii) V_{H}-C_{H2}; (iii) V_{H}-C_{H3}; (iv) V_{H}-C_{H1}-C_{H2}; (V) V_{H}-C_{H1}-C_{H2}-C_{H3}, (vi) V_{H}-C_{H2}-C_{H3}; (vii) V_{H}-C_{L}; (viii) V_{L}-C_{H1}; (ix) V_{L}-C_{H2}; (X) V_{L}-C_{H3}; (Xi) V_{L}-C_{H1}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

In one preferred embodiment of the invention, the antibody or antigen-binding fragment thereof is an anti-CD3 single-chain variable fragment (scFv).

In another preferred embodiment of the invention, the antibody or antigen-binding fragment thereof has an amino acid sequence of SEQ ID NO: 4 or a substantially similar sequence thereof; preferably having at least 90%, at least 95%, at least 98% or at least 99% sequence identity or a substantially similar sequence thereof.

The antibody disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes an antibody, and an antigen-binding fragment thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another mammalian germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes an anti-CD3 antibody comprising variants of any of the V_{H}, V_{L}, and/or CDR amino acid sequences disclosed herein having one or more conservative substitutions. For example, the present disclosure includes an anti-CD3 antibody having V_{H}, V_{L}, and/or CDR amino acid sequences with, e.g., 10 or fewer, 8 or fewer, 6 or fewer, 4 or fewer, etc. conservative amino acid substitutions relative to any of the V_{H}, V_{L}, and/or CDR amino acid sequences disclosed herein.

In one preferred embodiment of the disclosure, the extracellular domain of CD16 directly links to the antibody or antigen-binding fragment thereof. In another embodiment of the disclosure, a linker exists between the extracellular domain of CD16 and the antibody or antigen-binding fragment thereof.

In one preferred embodiment of the disclosure, the fusion protein further comprises a secretion signal peptide. The signal peptide (sometimes referred to as signal sequence, targeting signal, localization signal, localization sequence, transit peptide, leader sequence or leader peptide) as used herein refers to a short peptide located at the N-terminus of a protein that is destined towards the secretory pathway. In one embodiment of the disclosure, the secretion signal peptide has an amino acid sequence of SEQ ID NO: 6 or a substantially similar sequence thereof.

In one preferred embodiment of the disclosure, the fusion protein further comprises a protein purification tag.

In one preferred embodiment of the disclosure, the fusion protein has an amino acid sequence of SEQ ID NO: 8 or a substantially similar sequence thereof.

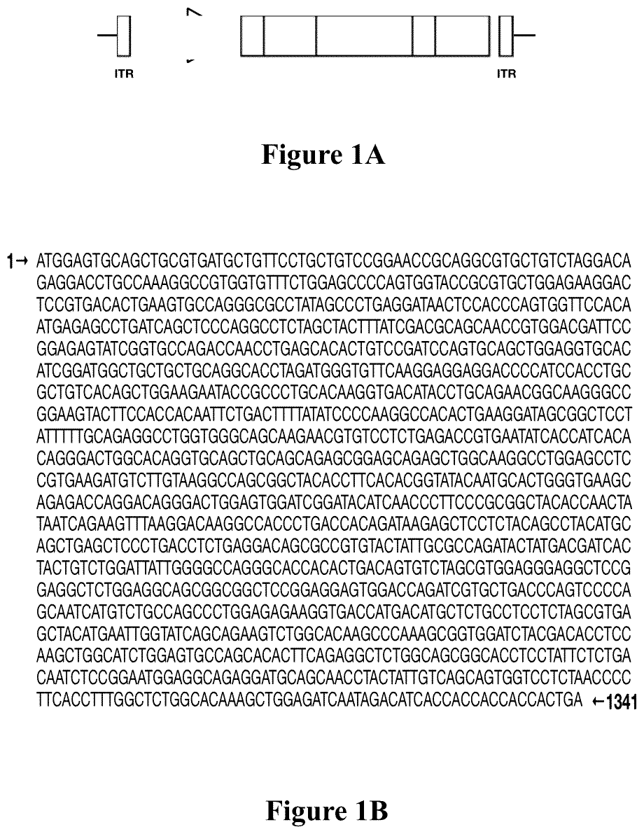

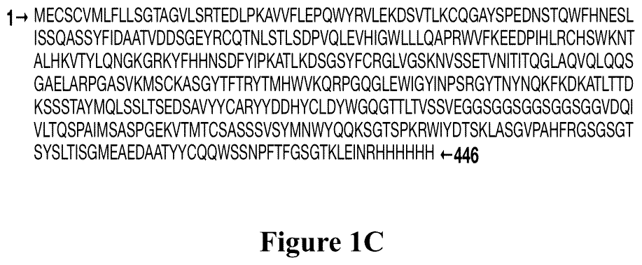

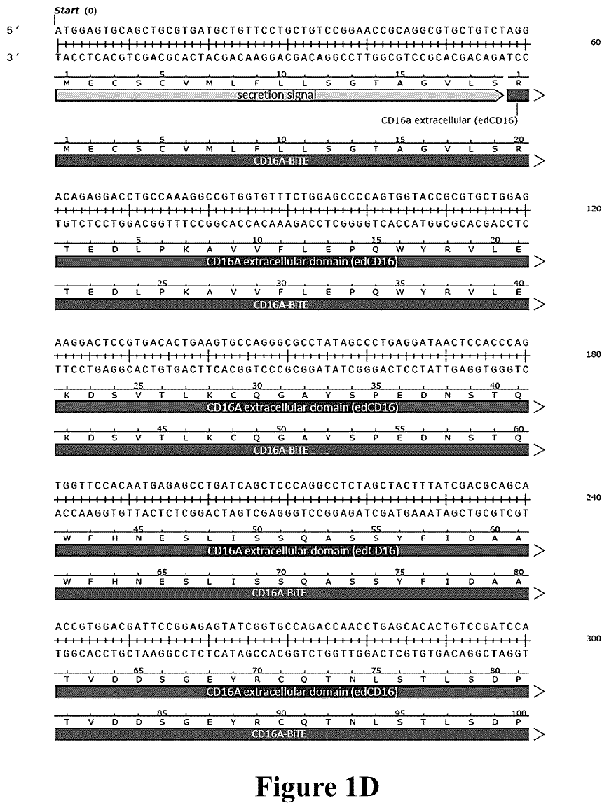

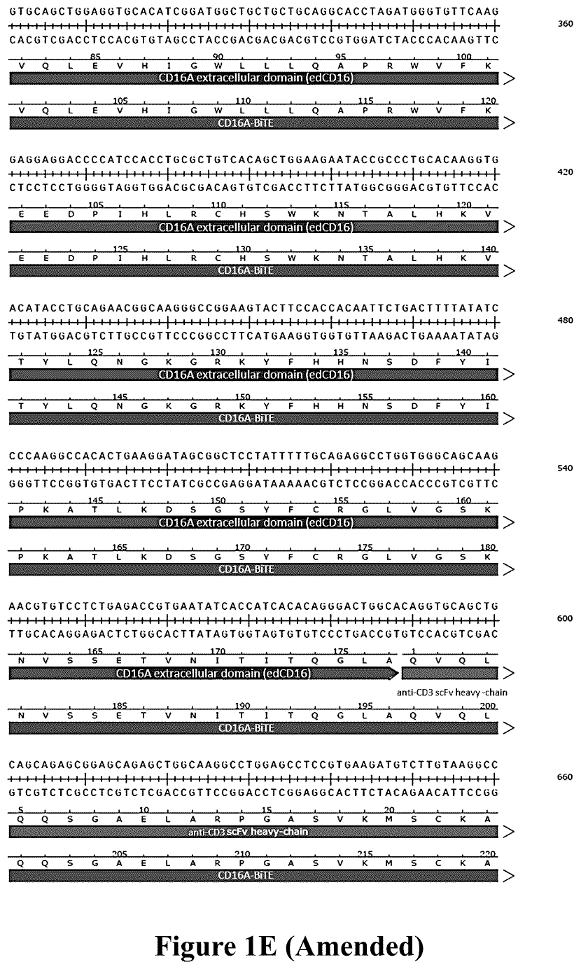

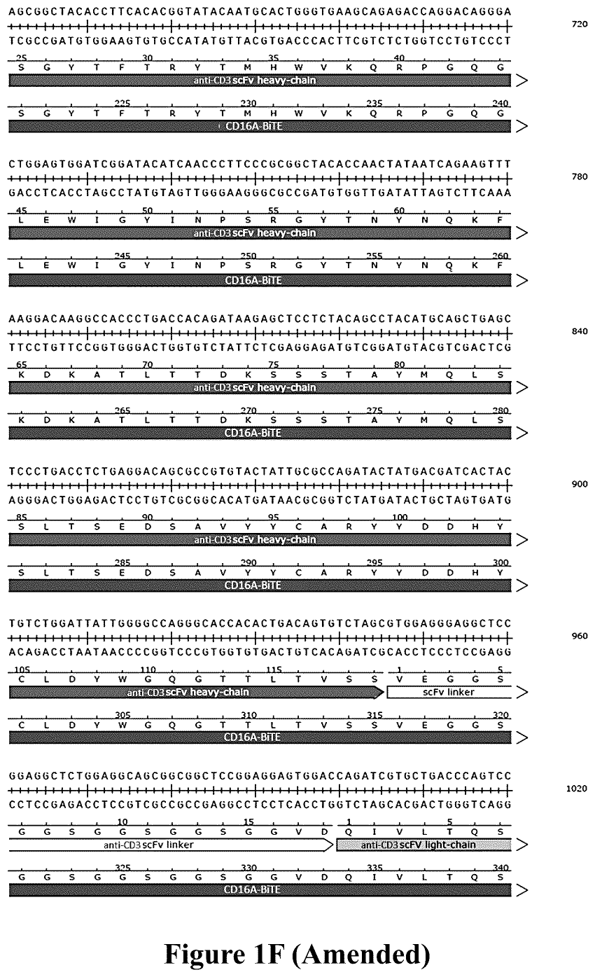

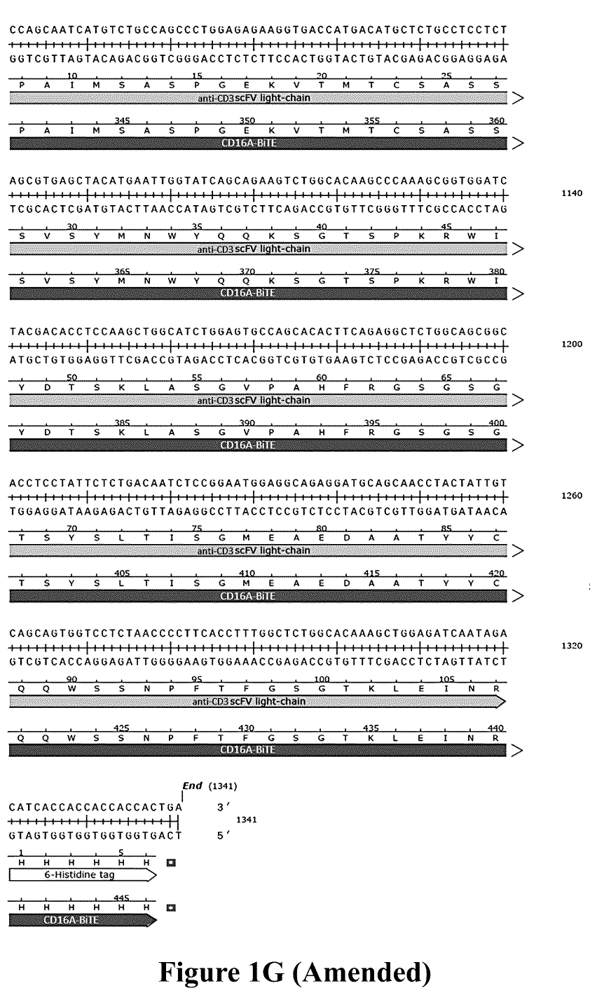

In another preferred embodiment of the disclosure, a novel bispecific T cell engager composed of the extracellular domain of the high-affinity CD16A variant and a single-chain anti-CD3 antibody (haCD16A-BiTE; FIG. 1A) effects the ADCC of mAbs in various fields of application. Such use of the haCD16A-BiTE provides several advantages: (1) overcoming limited availability of CD16A-expressing natural killer cells in the body, since with the haCD16A-BiTE, all of the CD3-expressing cells, including alpha-beta T cells, gamma-delta T cells, and natural killer T cells in the body could potentially be recruited, through the anti-CD3 portion of the haCD16A-BiTE binding to the CD3 molecules on T cells, to become cells carrying high-affinity CD16A and capable of carrying out ADCC; (2) resolving the issue of only 10-20% of general population expressing high-affinity CD16A variant which contributes to treatment efficacy of approved mAbs by applying the haCD16A-BiTE to any individual and consequently endowing large number of CD3⁺ T cells in each individual with high-affinity CD16A through the binding of the anti-CD3 portion of the haCD16A-BiTE to the CD3 molecules on T cells; (3) circumventing the down-regulation of CD16A on activated NK cells through adopting the extracellular domain of high-affinity CD16A in the design of this haCD16A-BiTE; (4) creating the feasibility of ADCC-mediated depletion of unwanted cells in T cell expansion culture prepared for immunotherapy by endowing T cells with ADCC activity with this haCD16A-BiTE; (5) providing a potential approach to create a viral vaccine with antibody-induced ADCC capability through the combined use of a vaccine and this haCD16A-BiTE and to increase treatment efficacy of antibody therapy of virally-infected diseases; (6) transforming suppression-removed T cells in patients receiving immune checkpoint inhibitor into T cells with ADCC activity to increase the therapeutic potential of avelumab-like anti-PD-L1 mAbs.

The present disclosure also provides a polynucleotide encoding the fusion protein as disclosed herein.

Preferably, the polynucleotide comprises a fragment encoding the extracellular domain of high-affinity CD16A and has a nucleic acid sequence of SEQ ID NO: 1 or a substantially identical sequence thereof.

Preferably, the polynucleotide comprises a fragment encoding the antibody or antigen-binding fragment thereof and has a nucleic acid sequence of SEQ ID NO: 3 or a substantially identical sequence thereof.

Preferably, the polynucleotide further comprises a fragment encoding a secretion signal peptide and has a nucleic acid sequence of SEQ ID NO: 5 or a substantially identical sequence thereof.

Preferably, the polynucleotide further comprises a fragment encoding a protein purification tag.

More preferably, the polynucleotide has a nucleic acid sequence of SEQ ID NO: 7 or a substantially identical sequence thereof.

The term “substantial identity” or “substantially identical,” when referring to a nucleic acid or a fragment thereof, indicates that, when optimally aligned with appropriate nucleotide insertions or deletions with another (refrence) nucleic acid (or its complementary strand), there is nucleotide sequence identity in at least 95%, and more preferably at least 96%, 97%, 98% or 99% of the nucleotide bases, to the entire sequence of said reference nucleic acid sequence as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

In one preferred embodiment of the disclosure, the fusion protein can be produced using any number of expression systems, including prokaryotic and eukaryotic expression systems. Many such systems are widely available from commercial suppliers. In one embodiment, the fusion protein may be expressed using a vector, wherein the polynucleotide encoding said fusion protein is operably linked to a promoter sequence. In one embodiment, the promoter is a constitutive promoter. In another embodiment, the promoter is an inducible promoter.

In one embodiment, the polynucleotide or vector is contained in a virus. In another embodiment, the virus is selected from the group consisting of a retrovirus, a lentivirus, an adenovirus, and an adeno-associated virus. In one preferred embodiment of the disclosure, the polynucleotide or vector is contained in an adeno-associated virus shuttle plasmid.

The present disclosure also provides a host cell comprising the polynucleotide as disclosed herein. In one embodiment, the host cell is a prokaryotic cell. In another embodiment, the host cell is an eukaryotic cell. In another embodiment, the host cell is a mammalian cell. In a preferred embodiment, the host cell is a human cell.

Preferably, the host cell comprises an adeno-associated virus vector which comprises the polynucleotide as disclosed herein.

The present disclosure also provides a pharmaceutical composition comprising a therapeutically effective amount of the fusion protein as disclosed herein and optionally a pharmaceutically acceptable carrier or excipient.

The present disclosure also provides a pharmaceutical composition comprising a therapeutically effective amount of the host cell according to the disclosure. A bispecific T cell engager generally has a very short serum half-life, around 2 hours. This results in the application of BITE® requiring continuous intravenous infusion, which procedure is inconvenient for human use. A virus is preferably used to mediate in vivo continuous production of BITE® to overcome this shortcoming.

The pharmaceutical compositions of the disclosure are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of fusion protein administered to a patient may vary depending upon various factors, including the age and the size of the patient, target disease, conditions, route of administration, and the like. The preferred dose is typically calculated according to body weight or body surface area. Depending on the severity of the condition, the frequency and the duration of the treatment can be adjusted. Effective dosages and schedules for administering the fusion protein may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Routes of administration include, but are not limited to, intradermal, intramuscular, intraperitoneal, intravenous, intratumorous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In one embodiment of the invention, a pharmaceutical composition of the present disclosure can be delivered intratumorous, subcutaneously or intravenously with a standard needle and syringe.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

In certain situations, the pharmaceutical composition can be delivered in an injectable preparation. The injectable preparations may include dosage forms for intratumorous, intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the pharmaceutical composition in a sterile aqueous medium or an oily medium conventionally used for injections. Examples of the aqueous medium for injections include physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. Examples of the oily medium include sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

The present disclosure also provides use of the pharmaceutical composition as disclosed herein in the manufacture of a medicament for inducing antibody-dependent cellular cytotoxicity in a subject in need.

Preferably, the present disclosure provides use of the pharmaceutical composition in the manufacture of a medicament for the treatment of a cancer, infectious disease, autoimmune disease, graft versus host disease, or post-transplantation lymphoproliferative disease in a subject in need.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause. As is understood by one skilled in the art, prevention or preventing need not achieve absolute (complete) block or avoidance of the conditions. Rather, prevention may achieve substantial (e.g., over about 50%) reduction or avoidance of the diseases or conditions to be prevented. Unless otherwise indicated herein, either explicitly or by implication, if the term “treatment” (or “treating”) is used without reference to possible prevention, it is intended that prevention be encompassed as well.

“Cancer”, “tumor”, “transformed” and like terms include precancerous, neoplastic, transformed, and cancerous cells, and can refer to a solid tumor, or a non-solid cancer (see, e.g., Edge et al. AJCC Cancer Staging Manual (7th ed. 2009); Cibas and Ducatman Cytology: Diagnostic principles and clinical correlates (3rd ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3rd ed. 1994)). Although transformation can arise from infection with a transforming virus and incorporation of new genomic DNA, or uptake of exogenous DNA, it can also arise spontaneously or following exposure to a carcinogen.

In one preferred embodiment of the disclosure, the pharmaceutical composition further comprises an antibody or antibodies. More preferably, the antibody is an IgG antibody. In another aspect, the antibody is a monoclonal antibody or polyclonal antibody. Examples of the antibody include but are not limited to an anti-CD20 antibody, anti-EGFR antibody, anti-HER2 antibody, anti-latent membrane protein 1 (LMP1) antibody, or anti-PD-L1 antibody.

Cancer development and progression are characterized by evasion of immune responses, including tumor escape mediated through immune checkpoint pathways (Pardoll Nat Rev Cancer, 12, 252-264 (2012)). By overexpressing PD-L1, tumor cells exploit the PD-1/PD-L1 pathway to promote an immunosuppressive environment and allow tumor growth (Topalian et al., Curr Opin Immunol, 24, 207-212 (2012)). Blocking PD-L1 inhibitory signals can restore T-cell anti-tumor activity and thus represents a key therapeutic strategy (Topalian et al., Curr Opin Immunol, 24, 207-212 (2012); Postow et al., J Clin Oncol, 33, 1974-1982 (2015)). Avelumab, an approved human IgG anti-PD-L1 mAb, is thought to specifically bind to PD-L1,preventing the interaction between PD-L1 and the inhibitory T-cell receptor PD-1. PD-L1 blockade removes the suppression of T-cell activity, resulting in T-cell-mediated anti-tumor immune responses (Hamilton and Rath Expert Opinion on Biological Therapy, 17, 515-523 (2017)). In addition, unlike other approved anti-PD-L1 antibodies, avelumab has a wild-type IgG Fc region, which enables avelumab to engage with FcγRs on NK cells and induce tumor-directed ADCC (Boyerinas et al., Cancer Immunol Res, 3, 1148-1157 (2015); Hamilton and Rath Expert Opinion on Biological Therapy, 17, 515-523 (2017)). Consequently, avelumab has the potential to both reactivate T cell-mediated antitumor immune responses and mediate eradication of tumor cells by ADCC. The phamaceutical composition according to the disclosure endows these suppression-removed T cells with ADCC activity and significantly increases the numbers of cell with ADCC activity and therapeutic potential of mAbs.

In one embodiment of the disclosure, the fusion protein is applied to delete unwanted cells. For example, to mitigate graft versus host disease and to prevent post-transplantation lymphoproliferative disease, such as post-transplantation Epstein-Barr virus (EBV)-induced lymphoproliferative disease.

In one embodiment of the disclosure, the fusion protein is applied to treat infectious diseases. According to the present disclosure, the infectious disease preferably includes viral infection, such as human immunodeficiency virus (HIV), Hepatitis B Virus (HBV), Epstein-Barr virus (EBV), and Cytomegalovirus (CMV). Not to be limited by theory, it is believed that ADCC is a potentially important protection mechanism in HIV vaccine (Haynes et al., N Engl J Med, 366, 1275-1286 (2012); Parsons et al., Retrovirology, 15, 58 (2018)). In addition, eliminating latently infected cells, which harbor the viral reservoir, is a major effort in HIV treatment. Many highly potent neutralizing antibodies that neutralize broad arrays of HIV-1 isolates, termed broadly neutralizing antibodies, have been isolated in recent years (Mujib et al., J Virol, 91, e00634-17 (2017)). Passive transfer of the broadly neutralizing antibody-VRC01 is currently under clinical evaluation for its potential to eliminate latently infected cells (NCT02716675 and NCT02568215) and there is a body of evidence suggesting NK cell-mediated ADCC lies in its potential ability to eliminate latently infected cells (Madhavi et al., J Virol, 91, e00700-17 (2017)). Nonetheless, chronic HIV-1 infection has been demonstrated to alter the phenotype, functionality and subset distribution of NK cells. Novel approaches to enhance ADCC, such as that disclosed herein, in HIV-infected patients could critically contribute to achieve a cure for HIV. Similar strategies could also be applied to vaccine design and treatment of other viral infections, such as HBV, EBV, and CMV (Gao et al., Human Vaccines Immunotherapeutics, 13, 1768-1773 (2017); Coghill et al., Clin Cancer Res, 22, 3451-3457 (2016); McVoy et al., Int J Mol Sci, 19, 3982 (2018)).

The following examples are provided to aid those skilled in the art in practicing the present disclosure.

### EXAMPLES

### Example 1

haCD16A-BITE Construct

The genetic construction of haCD16A-BiTE is shown in FIGS. 1A to 1G. FIG. 1A: The genetic construct of CD16A-CD3 bispecific T cell engager was cloned in an adeno-associated virus (AAV) shuttle plasmid pAAV-CD16CD3 and driven by the cytomegalovirus promoter (CMV) for the expression of a fusion protein with 446 amino acids. H: 6×Histidine tag; ITR: inverted terminal repeat sequence of AAV; S: secretion signal; WPRE: woodchuck hepatitis B virus post-transcriptional regulatory element. FIG. 1B: The coding sequences of the extracellular domain (edCD16) of human high-affinity CD16A and a single-chain antibody against human CD3 (anti-CD3 scFv) were fused in the same coding frame by gene synthesis. FIG. 1C: The synthesized gene with 1341 bp nucleotides encodes a fusion protein with 446 amino acids. FIG. 1D to 1G: Scheme of haCD16A-BiTE.

In vitro Binding Assay of the haCD16A-BiTE

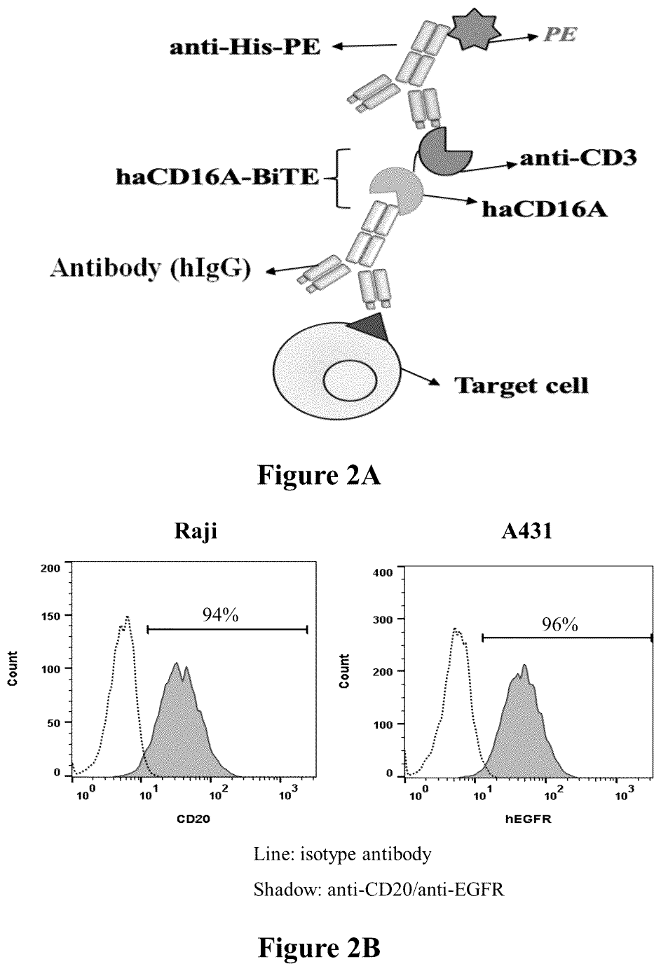

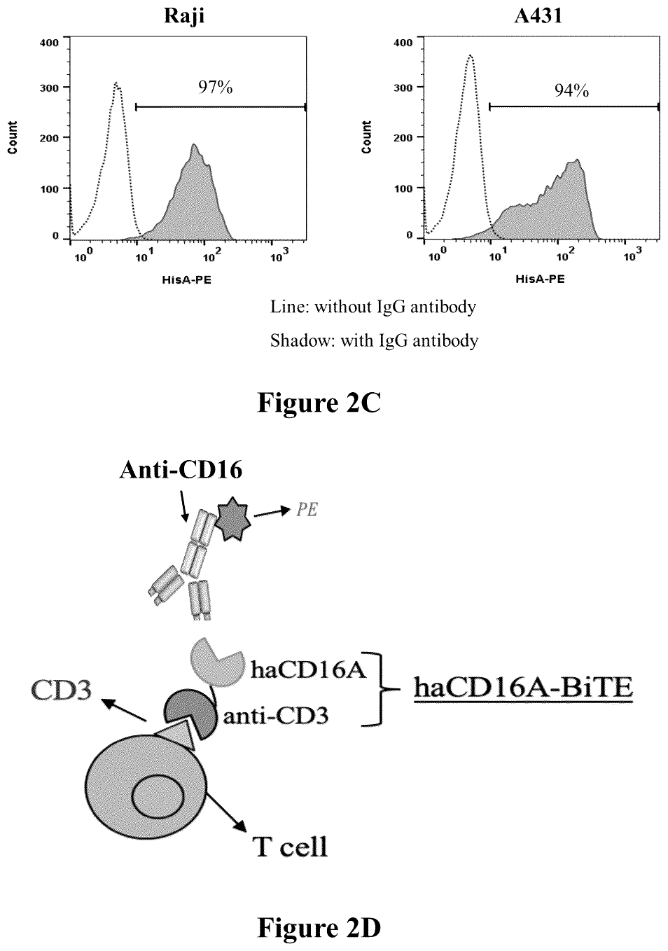

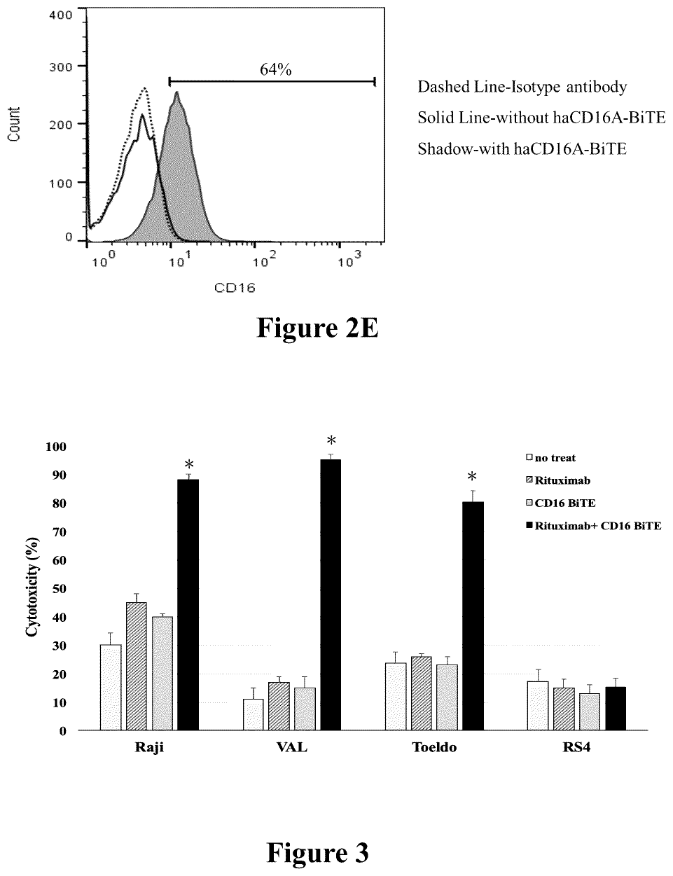

The haCD16A-BiTE has the ability to bind human IgG antibodies and T cells. This binding capability of the haCD16A-BiTE was first demonstrated. The haCD16A-BiTE with histidine-tags were produced and purified from supernatants of the haCD16A-BiTE gene-transfected HEK 293 cells by affinity chromatography. To demonstrate the binding of the haCD16A-BiTE to IgG antibodies, the analytical strategy is outlined in FIG. 2A. Raji (CD20⁺ Burkett lymphoma cells) and A431 (EGFR⁺ epidermoid carcinoma cells) cells were first incubated with 1·g of rituximab and cetuximab (both are IgG antibodies) respectively for 10 minutes and then with 50 ng of the haCD16A-BiTE in 100 μl of PBS at 4° C. for 10 minutes. Next, cells were pelleted by centrifugation at 400×g for 5 minutes, washed once with PBS, and incubated with 2 μl of a PE-labeled anti-6x-histidine tag antibody (Miltenyi Biotech) in 100 μl of PBS at 4° C. for 1 hour. After washing once with PBS, cells were subjected to flow cytometry analysis, and the data were expressed as a histogram with counts versus fluorescence. Raji and A431 cells were also examined for CD20 and EGFR expression by flow cytometry.

As shown in FIG. 2B, Raji and A431 cells expressed CD20 and EGFR, respectively. In the presence of the haCD16A-BiTEs, more than 90% of Raji or A431 cells treated with rituximab or anti-EGFR antibodies were stained positive with the anti-6×-histidine tag (anti-His) antibody, but not untreated cells (FIG. 2C), indicating the haCD16A-BiTE has the ability to bind IgG antibody. These experimental data demonstrate the haCD16A-BiTEs are able to bind to IgG antibody-coated tumor cells.

To show the binding of the haCD16A-BiTE to T cell, FIG. 2D illustrates the analytical strategy and FIG. 2E shows CD16-negative T cells, more than 95% of which expressing CD3 molecules, were detected by anti-CD16 antibody in the presence of the haCD16A-BiTEs, but not without the haCD16A-BiTEs. These results indicate the haCD16A-BiTE binds to T cells. The experimental methods used were as above described, except CD16-T cells were used and IgG antibodies were omitted.

### Example 2

In Vitro Activity Assay of the haCD16A-BiTE
Ability of the haCD16A-BiTE to Mediate CD20-Expressing Tumor Cell Killing in the Presence of Approved Anti-CD20 IgG Antibody (Rituximab) and T Cells

The cytotoxicity experiment was carried out in accordance with the method disclosed in Sheehy et al. (J Immunol Methods, 249, 99-110 (2001)). In this experiment, CD20-expressing Raji, VAL, and Toeldo blood tumor cell lines were used as target cells, wherein the VAL cell line was acute lymphocytic leukemia (ALL) cells, and Toeldo cell line was diffuse large B cell lymphoma cells. The RS4 cell line was CD20⁻ ALL cells. Raji, VAL, Toeldo, and RS4 cells were independently stained with 5 (6)-carboxyfluorescein diacetate succinimidyl ester (CFSE) and seeded in the wells of culturing plates (5×10^{4/}well). The haCD16A-BiTEs (80 ng/well), rituximab (10 μg/well), and T cells (5×10⁵ cells/well) were added independently or together into each of the wells containing different tumor cells. After cultures were incubated for 6 hours, the viability of the cells was determined by counting CFSE⁺ cells using flow cytometry. T cells were obtained from peripheral blood mononuclear cells (PBMC) cultures expanded with CD3/CD28 beads, IL-7, and IL-15, according to methods described by Chen et al. (Clinical Immunology, 104, 58-66, (2002)).

The results as shown in FIG. 3 prove that, in the presence of T cells, the haCD16A-BiTE exhibits a synergistic effect in killing CD20-expressing blood tumor cells (Raji, VAL and Toeldo) when combined with rituximab, as compared to using the haCD16A-BiTE or rituximab alone. *: p<0.001, compared to the haCD16A-BiTE or rituximab alone.

Ability of the haCD16A-BiTE to Mediate EGFR-Expressing Tumor Cell Killing in the Presence of Approved Anti-EGFR IgG Antibodies (Cetuximab) and T cells

The experiment was carried out exactly as described in FIG. 3, except that EGFR-expressing tumor cell line (A431) and cetuximab (2.5 μg/well) were used instead. In this experiment, A431 cells expressing EGFR molecules were used as target cells. The MCF-7 cell line was EGFR-low breast tumor cells.

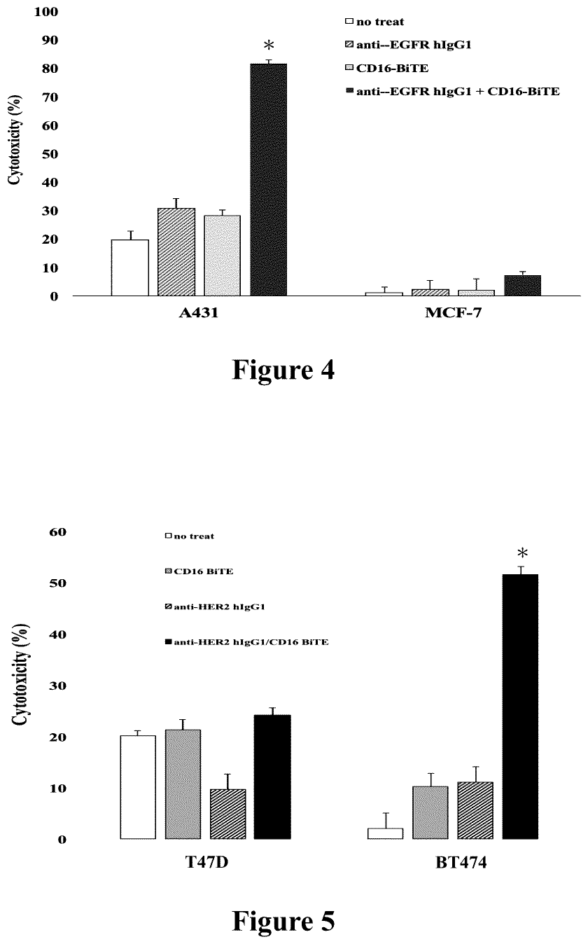

The results as shown in FIG. 4 prove that in the presence of T cells, the haCD16A-BiTE exhibits a synergistic effect in killing EGFR-expressing cells (A431) when combined with cetuximab, as compared to using the haCD16A-BiTE or cetuximab alone. *: p<0.001, compared to the haCD16A-BiTE or cetuximab alone.

Ability of the haCD16A-BiTE to Mediate HER2-Expressing Tumor Cell Killing in the Presence of Approved Anti-HER2 IgG Antibodies (Trastuzumab) and T cells

The experiment was carried out exactly as described in FIG. 3, except that HER2-expressing tumor cell line and trastuzumab (2.5 μg/well) were used instead. In this experiment, BT474 tumor cell line expressing HER2 molecules in >80% of cells were used as target cells, wherein BT474 was breast ductal carcinoma cell. The T47D cell line was HER2-low mammary gland ductal carcinoma cells with 10% of cells expressing HER2 in low density.

The results as shown in FIG. 5 prove that in the presence of T cells, the haCD16A-BiTE exhibits a synergistic effect in killing HER2-high cells (BT474 cells), but not HER2-low cells (T47D cells) when combined with trastuzumab, as compared to using the haCD16A-BiTE or trastuzumab alone. *: p<0.01, compared to the haCD16A-BiTE or trastuzumab alone.

Altogether, experimental data as shown in Example 2 demonstrate that the haCD16A-BiTE is able to recruit T cells to mediate killing of IgG antibody-bound tumor cells. Importantly, these results also demonstrate that haCD16A-BiTE is able to combined with various FDA-approved IgG therapeutic antibodies to kill tumor cells.

### Example 3

The Effect of Blood Plasma on the Activity of the haCD16A-BiTE

Normal plasma levels of IgG may compete with therapeutic mAbs of IgG isotype for binding to the haCD16A-BiTE resulting in loss of the haCD16A-BiTE activity. To evaluate this competition, whole blood from healthy individuals was centrifuged at 400×g for 5 minutes and the supernatants were taken as blood plasma. Plasma was added, at different volume ratios, to the serum-free medium used for cytotoxicity assay experiments illustrated in FIG. 3 to analyze the effect of plasma on the cytotoxicity of rituximab in the presence of the haCD16A-BiTE and T cells. Plasma also mediates complement-dependent cytotoxicity of rituximab, thus data were expressed by subtracting out that of the cytotoxicity of rituximab/plasma/T cells.

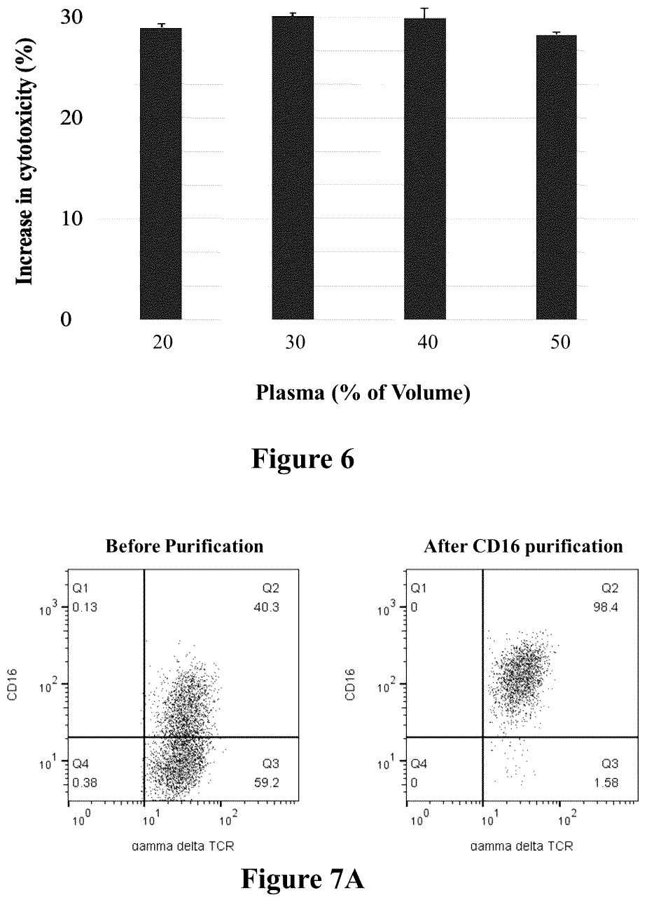

FIG. 6 shows that blood plasma, up to 50%, did not significantly reduce the cytotoxicity of rituximab on Raji cells in the presence of the haCD16A-BiTE and T cells.

### Example 4

Comparison Study of IgG antibody-Mediated Cell Killing Between CD16⁺γ9δ2 T Cells and CD16⁻γ9δ2 T Cells Pulsed with the haCD16A-BITEs

To evaluate the equivalence of the haCD16A-BiTEs to the high-affinity CD16A expressed on T cells, we compared the antibody-mediated cytotoxicity of CD16⁻γ9δ2 T cells pulsed with the haCD16A-BiTEs with that of CD16⁺γ9δ2 T cells having a high affinity CD16 variant.

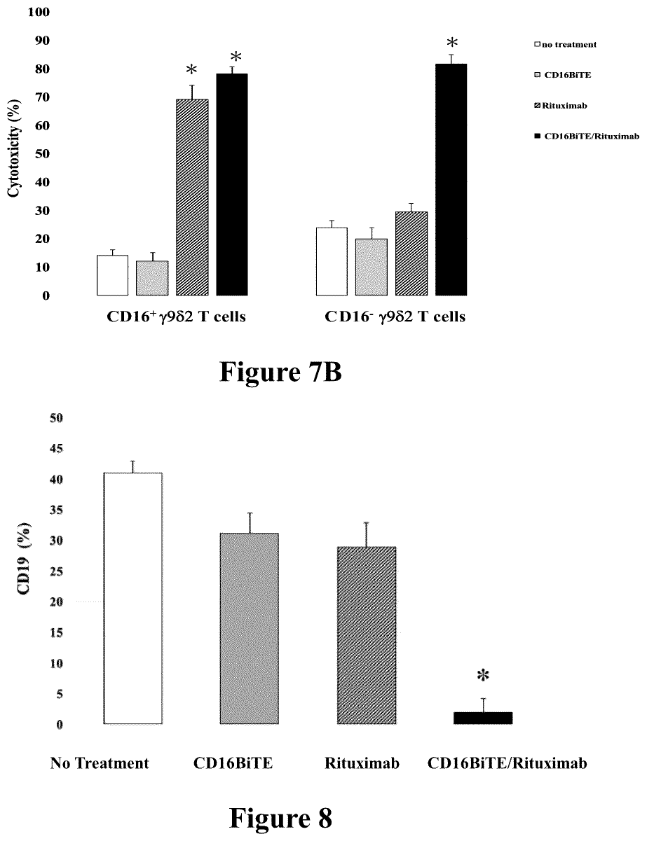

γ9δ2 T cells were generated as described below. PBMC (2×10⁶ cells/ml) from different donors were stimulated with recombinant human IL2 (25 ng/ml; Prospec) and Zoledronate (1 μM; Sigma) for 14 days in RPMI-1640 medium containing 10% heat-inactivated FBS, penicillin (100 IU/ml), and streptomycin (100 μg/ml) at 37° C. in a humidified incubator with 5% CO₂. Subsequently, γ9δ2 T cell cultures were analyzed for CD16 expression and for those cultures stained positive for CD16, a polymerase chain reaction specific for CD16 high-affinity variant was performed to select high-affinity variant cultures of CD16⁺-γ9δ2 T cells. CD16⁺-γ9δ2 T cells were purified with an anti-PE purification kit from Miltenyi Biotech coupling with an anti-CD16 antibody labelled with PE according to the manufacturer's instruction and >95% of these purified γ9δ2 T cells express CD16 (FIG. 7A). Those cultures stained negative for CD16 expression were used as cell sources of CD16⁻-γ9δ2 T cells.

Antibody-mediated cell killing were analyzed according to methods described in Example 2 using rituximab and Raji cell as target cell. To study antibody-mediated cell killing of CD16⁺-γ9δ2 T cells, Raji were stained with CFSE and seeded in the wells of culturing plates (5×10^{4/}well) with rituximab (20 μg/well) and CD16⁺-γ9δ2 T cells (5×10⁵ cells/well). To evaluate antibody-mediated cell killing of CD16⁻-γ9δ2 T cells in combination with the haCD16A-BiTEs, Raji cells stained with CFSE (5×10^{4/}well), haCD16A-BiTE (80 ng/well), Rituximab (20 μg/well), and CD16⁻-γ9δ2 T cells (5×10⁵ cells/well) were added together into the wells. After cultures being incubated for 6 hours, the viability of Raji cells was determined by counting CFSE⁺ cells using flow cytometry.

Results displayed in FIG. 7B show that rituximab-mediated Raji cell killing of CD16⁻-γ9δ2 T cells in combination with the haCD16A-BiTE was comparable to that of CD16⁺-γ9δ2 T cells, indicating that the functional activity of the haCD16A-BiTE is equvalent to that of high-affinity CD16 variants expressed on γ9δ2 T cells. *: p<0.001, compared to the haCD16A-BiTE or rituximab alone for CD16⁻-γ9δ2 T cells. *: p<0.001, compared to the haCD16A-BiTE alone for CD16⁺-γ9δ2 T cells.

### Example 5

The use of the haCD16A-BiTE to Deplete Unwanted Cells from T Cell Expansion Culture

This example is to demonstrate the usefulness of the haCD16A-BiTE in depleting unwanted cells, such as tumor cells, from T cell expansion culture.

Written informed consent agreement were obtained from patients with chronic lymphocytic leukemia (CLL) and then peripheral blood samples were obtained. PBMC were isolated from 5 ml venous blood by density gradient centrifugation using FICOLL® Paque PLUS (Sigma) according to the manufacturer's instruction. For the expansion of γ9δ2 T cells, PBMC (2×10⁶ cells/ml) were stimulated with recombinant human IL2 (25 ng/ml; Prospec) and Zoledronate (1 μM; Sigma) for 14 days in RPMI-1640 medium containing 10% heat-inactivated FBS, penicillin (100 IU/ml), and streptomycin (100 μg/ml) at 37° C. in a humidified incubator with 5% CO₂. To deplete malignant B cells from these T cell expansion cultures, the haCD16A-BiTEs (80 ng/well) and rituximab (10 μg/well) were added to the cultures at day 10. Three days later, cultures were analyzed for malignant B cell death by flow cytometry analysis of both CD19 marker (a marker for malignant B cell) and propidium iodide (PI) staining.

FIG. 8 shows that CD19-positive cells decreased after treatment with the haCD16A-BiTEs and rituximab, demonstrating that the haCD16A-BiTE combined with rituximab has the ability to deplete malignant B cells from T cell expansion culture. *: p<0.001, compared to the haCD16A-BiTE or rituximab alone.

### Example 6

The Ability of the haCD16A-BiTE to Mediate Killing of EBV-Infected B Cells in the Presence of Anti-EBV IgG Antibodies and T Cells

To establish the usefulness of the haCD16A-BiTE in viral disease treatment, EBV-infected B cell lines were used as model virally-infected cells to test the ability of the haCD16A-BiTE to mediate killing of EBV-infected B cells in the presence of anti-EBV IgG antibodies and T cells.

EBV is a gamma herpes virus that infects 90% of the population. EBV establishes life-long latency in memory B cells and oral epithelial cells. In the immune competent host, circulating EBV-specific cytotoxic T lymphocytes maintain EBV-infected B cells at a level of less than 1% of the B-cell pool. However, in the immunosuppressed host, uncontrolled proliferation of EBV-infected B cells contributes to lymphoproliferative disorders such as post-transplantation lymphoproliferative disorder (PTLD) after solid organ transplantation or hematopoietic stem cell transplantation. Moreover, a significant proportion of Hodgkin lymphomas, non-Hodgkin lymphomas, and nasopharyngeal carcinomas are associated with EBV infection. There are limited treatment options for EBV-related malignancies beyond standard chemotherapy and radiation. One promising modality is adoptive cell therapy using EBV-specific T cells, which has shown efficacy for PTLD (success in 70% of cases). A more ideal therapeutic agent would be an antibody that can specifically recognize one of the more broadly expressed antigens: latent membrane protein-1 (LMP1) or LMP2 (Ahmed et al. JCI Insight, 3, e97805, (2018)).

Immortalized EBV-infected B cells express LMP1 and were used as target cells and jurkat T cells as negative control cells. Experiments were performed exactly as described in Example 2, except that a human anti-LMP1 IgG antibody (10 μg/well) (Creative Biolab) were used instead.

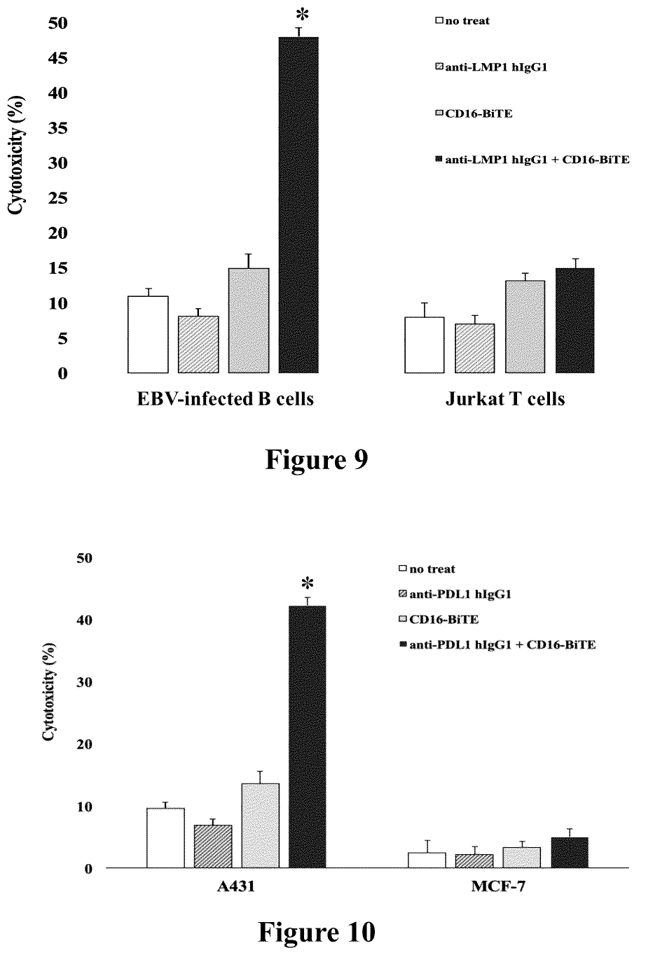

FIG. 9 prove that in the presence of T cells, the haCD16A-BiTE exhibits a synergistic effect in killing EBV-infected B cells when combined with the anti-LMP1 IgG antibody, as compared to using the haCD16A-BiTE or anti-LMP1 antibody alone. *: p<0.001, compared to the haCD16A-BiTE or anti-LMP1 antibody alone.

### Example 7

In vitro Activity Assay of the haCD16A-BiTE to Mediate Killing of PD-L1-Expressing Tumor Cells

By overexpressing PD-L1, tumor cells exploit the PD-1/PD-L1 pathway to promote an immunosuppressive environment and allow tumor growth (Topalian et al., Curr Opin Immunol, 24, 207-212 (2012)). Hence, PD-L1 also is a tumor-associated antigen. Blocking PD-L1 inhibitory signals with anti-PD-L1 IgG mAbs not only can restore T-cell anti-tumor activity, but also provide an opportunity to eradicate tumor cells by ADCC.

To examine the potential of the haCD16A-BiTE to endow T cells with ADCC activity to mediate eradication of PD-L1-expressing tumor cells by anti-PD-L1 IgG mAbs. We screened tumor cells for PD-L1-expression and carried out experiments exactly as described in Example 2, except that a PD-L1-expressing tumor cell line (A431) and a human anti-PD-L1IgG antibody (10 μg/well) (Invivogen) were used instead. In this experiment, A431 cells were used as target cells. The MCF-7 cell line was PD-L1-low breast cancer cells.

The results as shown in FIG. 10 prove that in the presence of T cells, the haCD16A-BiTE exhibits a synergistic effect in killing PD-L1-expressing cells (A431) when combined with an anti-PD-L1 IgG antibody, as compared to using the haCD16A-BiTE or anti-PD-L1 antibody alone. *: p<0.001, compared to haCD16A-BiTE or anti-PD-L1 antibody alone.

### Example 8

In Vivo Assay for the Activity of the haCD16A-BiTE on Killing of CD20-Expressing Tumor Cells in an Immunodeficient Nod Mouse Model

Our invention to be effective in enhancing efficacy of therapeutic antibodies requires the coming together of four components to form a complex. These components include the tumor cell, the antibody, the haCD16A-BiTE, and the T cell. In vivo proof-of-concept studies were performed to demonstrate that this complex formation occurs in vivo, in addition to demonstrating the in vivo activity of the haCD16A-BiTE.

These experiments were carried out in T cells-lacking immunodeficient NOD.Cg-Prkdc^{scid}Il2rg^{tm1Wjl}/YckNarl (RMRC 13288) mice. First, the genes of luciferase and green fluorescent protein were transduced into Raji blood tumor cells using lentiviral vector (Zhou et al., Blood, 120, 4334-4342 (2012).) The Raji cells (1×10⁶ cells) that express luciferase and green fluorescent protein were implanted into NOD mice via tail vein injection. On day 7 after the implantation of Raji cells, T cells obtained as described in Example 2 were implanted into the NOD mice via tail vein injection. The amount of T cells implanted per injection was 10-fold the amount of Raji cells. T cells were implanted once every 4 days for a total of 2 implantations. In addition, on day 7 after the implantation of Raji cells, the haCD16A-BiTE was infused via tail vein for 9 consecutive days with a daily dose of 800 ng, which was delivered by one bolus injection. Rituximab (10 mg/kg) was administered twice via tail vein on day 7 and 11 after Raji cell implantation. Mice of the control group received T cell implantation only.

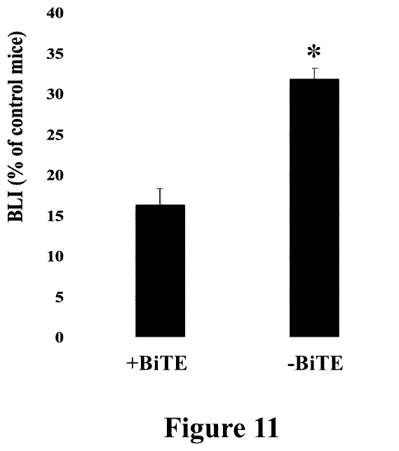

The bioluminescence imaging (BLI) was conducted at different time points to monitor the clearance of Raji cells. At the end of the experiment, body weights in the treatment groups were comparable to those of the controls. The results in FIG. 11 show the BLI difference at the end of the experiment. Compared to mice treated with rituximab and T cells (without haCD16A-BiTE), the haCD16A-BiTE significantly enhanced Raji cell clearance by two-fold. Accordingly, tumor cell, antibody, the haCD16A-BiTE, and T cell can combine together as required for activity in vivo. More importantly, these data demonstrate the in vivo activity of the haCD16A-BITE. *: p<0.01, compared to the haCD16A-BiTE, rituximab, and T cell combination.

### Example 9

AAV-haCD16A-BiTE Mediates Peripheral Expression of the haCD16A-BiTE in Mice

To overcome the requirement of continuous infusion of BiTEs in therapeutic settings due to their short in vivo half-life, a AAV-mediated gene transfer approach was explored.

Mice (B6 strain, male, 8-week old; n=5) were intraperitoneally injected with a viral vector of AAV-haCD16A-BiTE (FIG. 1A, 10⁹ vgc/animal; vgc: viral genome copy). Serum samples were collected from tail veins before (day 0) and after virus injection (day 2, 7, 14) and subjected to Western blot analysis to evaluate the protein levels of the haCD16A-BiTE. An HRP-conjugated polyclonal antibody was used to probe the 6×Histidine tag on the c-terminal of the haCD16A-BiTE. The levels of detected proteins were measured by densitometric analysis and presented as optical density values per ml of serum.

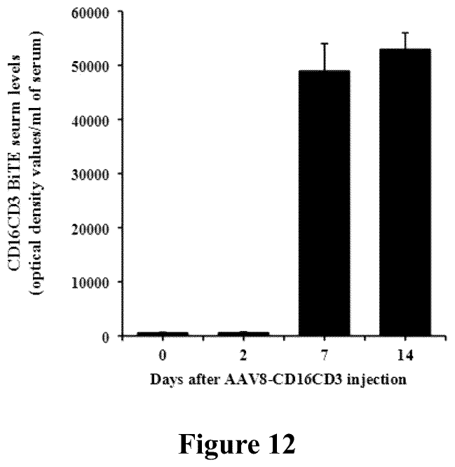

The results in FIG. 12 show that 7 days after injecting with AAV-haCD16A-BiTE, the haCD16A-BiTE was detected in peripheral blood of mice and its presence lasts for at least 7 days. These results demonstrate that in vivo persistent production of the haCD16A-BiTE is achievable by in vivo virus-mediated haCD16A-BiTE gene transfer.

While the present invention has been described in conjunction with the specific embodiments set forth above, many alternatives thereto and modifications and variations thereof will be apparent to those of ordinary skill in the art. All such alternatives, modifications and variations are regarded as falling within the scope of the present invention.

What is claimed is:

1. A fusion protein comprising:
an extracellular domain of human CD16A; and
an antibody or antigen-binding fragment thereof that specifically binds to an epitope on human CD3 or a fragment thereof.

2. The fusion protein according to claim 1, wherein the CD16A is a high-affinity CD16A variant.

3. The fusion protein according to claim 1, wherein the extracellular domain of CD16A has an amino acid sequence of SEQ ID NO: 2 or a substantially similar sequence thereof.

4. The fusion protein according to claim 1, wherein the antibody or antigen-binding fragment thereof is an anti-CD3 single-chain variable fragment.

5. The fusion protein according to claim 1, wherein the antibody or antigen-binding fragment thereof has an amino acid sequence of SEQ ID NO: 4 or a substantially similar sequence thereof.

6. The fusion protein according to claim 1, wherein the extracellular domain of CD16A directly links to the antibody or antigen-binding fragment thereof.

7. The fusion protein according to claim 1, which further comprises a secretion signal peptide.

8. The fusion protein according to claim 7, wherein the secretion signal peptide has an amino acid sequence of SEQ ID NO: 6 or a substantially similar sequence thereof.). The fusion protein according to claim 1, which further comprises a protein purification tag.

10. The fusion protein according to claim 1, which has an amino acid sequence of SEQ ID NO: 8 or a substantially similar sequence thereof.

11. A polynucleotide encoding the fusion protein according to claim 1.

12. The polynucleotide according to claim 11, which comprises a fragment encoding the extracellular domain of CD16A and has a nucleic acid sequence of SEQ ID NO: 1 or a substantially identical sequence thereof.

13. The polynucleotide according to claim 11, which comprises a fragment encoding the antibody or antigen-binding fragment thereof and has a nucleic acid sequence of SEQ ID NO: 3 or a substantially identical sequence thereof.

14. The polynucleotide according to claim 11, which further comprises a fragment encoding a secretion signal peptide and has a nucleic acid sequence of SEQ ID NO: 5 or a substantially identical sequence thereof.

15. The polynucleotide according to claim 11, which further comprises a fragment encoding a protein purification tag.

16. The polynucleotide according to claim 11, which has a nucleic acid sequence of SEQ ID NO: 7 or a substantially identical sequence thereof.

17. The polynucleotide according to claim 11, which is contained in an adeno-associated virus vector.

18. A host cell comprising the polynucleotide according to claim 11.

19. The host cell according to claim 18, wherein the polynucleotide is contained in an adeno-associated virus vector.

20. A pharmaceutical composition comprising a therapeutically effective amount of the fusion protein according to claim 1 and optionally a pharmaceutically acceptable carrier or excipient.

21. The pharmaceutical composition according to claim 20, which further comprises an antibody or antibodies.

22. Use of the pharmaceutical composition according to claim 20 in the manufacture of a medicament for inducing antibody-dependent cellular cytotoxicity in a subject in need.

23. Use of the pharmaceutical composition according to claim 21 in the manufacture of a medicament for inducing antibody-dependent cellular cytotoxicity in a subject in need.

24. Use of the pharmaceutical composition according to claim 20 in the manufacture of a medicament for the treatment of a cancer, an infectious disease, an autoimmune disease, a graft versus host disease, or a post-transplantation lymphoproliferative disease in a subject in need.

25. Use of the pharmaceutical composition according to claim 21 in the manufacture of a medicament for the treatment of a cancer, an infectious disease, an autoimmune disease, a graft versus host disease, or a post-transplantation lymphoproliferative disease in a subject in need.

1-25. (canceled)

26. A method for inducing antibody-dependent cellular cytotoxicity directed against tumor cells or virally-infected cells in a subject having a tumor comprising said tumor cells or having said virally-infected cells, comprising administrating to the subject:
a pharmaceutical composition comprising an effective amount of an isolated fusion protein, wherein the fusion protein comprises
(i) an Fc-binding extracellular domain of human CD16A, wherein the Fc-binding extracellular domain of human CD16A has the amino acid sequence as set forth in SEQ ID NO: 2; and
(ii) a known anti-human CD3 single-chain variable fragment that both specifically binds to human CD3 and activates human T cells; and
wherein the C-terminal residue of SEQ ID NO: 2 is directly fused to the N-terminal residue of the anti-human CD3 single-chain variable fragment; and
optionally a pharmaceutically acceptable carrier or excipient.

27. The method of claim 26, wherein the pharmaceutical composition further comprises an antibody comprising a human IgG Fc domain, which specifically binds to an antigen on the surface of said tumor or virally-infected cells.

28. The method of claim 26, wherein the Fc-binding extracellular domain of human CD16A consists of the amino acid sequence of SEQ ID NO: 2.

29. The method of claim 26, wherein said antibody specifically binds to a tumor antigen selected from CD20, EGFR, HER2, and PD-L1, or to an EBV antigen selected from LMP1 and LMP2.

30. A method for treating cancer or viral infection in a subject having cancer or viral infection by inducing antibody-dependent cellular cytotoxicity directed against the cancer cells or virally-infected cells in the subject, said method comprising administrating to the subject:
a pharmaceutical composition comprising a therapeutically effective amount of an isolated fusion protein, wherein the fusion protein comprises
(i) an Fc-binding extracellular domain of human CD16A, wherein the Fc-binding extracellular domain of human CD16A has the amino acid sequence as set forth in SEQ ID NO: 2; and
(ii) a known anti-human CD3 single-chain variable fragment that both specifically binds to human CD3 and activates human T cells; and
wherein the C-terminal residue of SEQ ID NO: 2 is directly fused to the N-terminal residue of the anti-human CD3 single-chain variable fragment; and
optionally a pharmaceutically acceptable carrier or excipient.

31. The method of claim 30, wherein the pharmaceutical composition further comprises an antibody comprising a human IgG Fc domain, which specifically binds to an antigen on the surface of said tumor or virally-infected cells.

32. The method of claim 30, wherein the Fc-binding extracellular domain of human CD16A consists of the amino acid sequence of SEQ ID NO: 2.

33. The method of claim 30, wherein said antibody specifically binds to a tumor antigen selected from CD20, EGFR, HER2, and PD-L1, or to an EBV antigen selected from LMP1 and LMP2.

34. The method of claim 30, wherein said subject is human.


SEQUENCE LISTING (8 sequences)

SEQ ID NO: 1 - DNA, 531 bp, Homo sapiens
  source 1..531: mol_type = other DNA
  CDS 1..531
aggacagaggacctgccaaaggccgtggtgtttctggagccccagtggtaccgcgtgctggagaaggactccgtgacactgaagtgccagggcgcctatagccctgaggataactccacccagtggttccacaatgagagcctgatcagctcccaggcctctagctactttatcgacgcagcaaccgtggacgattccggagagtatcggtgccagaccaacctgagcacactgtccgatccagtgcagctggaggtgcacatcggatggctgctgctgcaggcacctagatgggtgttcaaggaggaggaccccatccacctgcgctgtcacagctggaagaataccgccctgcacaaggtgacatacctgcagaacggcaagggccggaagtacttccaccacaattctgacttttatatccccaaggccacactgaaggatagcggctcctatttttgcagaggcctggtgggcagcaagaacgtgtcctctgagaccgtgaatatcaccatcacacagggactggca

SEQ ID NO: 2 - protein, 177 aa, Homo sapiens
RTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLA

SEQ ID NO: 3 - DNA, 732 bp, synthetic construct
  misc_feature 1..732: note = scFv
  source 1..732: mol_type = other DNA
  CDS 1..732
caggtgcagctgcagcagagcggagcagagctggcaaggcctggagcctccgtgaagatgtcttgtaaggccagcggctacaccttcacacggtatacaatgcactgggtgaagcagagaccaggacagggactggagtggatcggatacatcaacccttcccgcggctacaccaactataatcagaagtttaaggacaaggccaccctgaccacagataagagctcctctacagcctacatgcagctgagctccctgacctctgaggacagcgccgtgtactattgcgccagatactatgacgatcactactgtctggattattggggccagggcaccacactgacagtgtctagcgtggagggaggctccggaggctctggaggcagcggcggctccggaggagtggaccagatcgtgctgacccagtccccagcaatcatgtctgccagccctggagagaaggtgaccatgacatgctctgcctcctctagcgtgagctacatgaattggtatcagcagaagtctggcacaagcccaaagcggtggatctacgacacctccaagctggcatctggagtgccagcacacttcagaggctctggcagcggcacctcctattctctgacaatctccggaatggaggcagaggatgcagcaacctactattgtcagcagtggtcctctaaccccttcacctttggctctggcacaaagctggagatcaataga

SEQ ID NO: 4 - protein, 244 aa, synthetic construct
  REGION 1..244: note = Synthetic Construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 5 - DNA, 57 bp, synthetic construct
  misc_feature 1..57: note = secretion signal
  source 1..57: mol_type = other DNA
  CDS 1..57
atggagtgcagctgcgtgatgctgttcctgctgtccggaaccgcaggcgtgctgtct

SEQ ID NO: 6 - protein, 19 aa, synthetic construct
  REGION 1..19: note = Synthetic Construct
MECSCVMLFLLSGTAGVLS

SEQ ID NO: 7 - DNA, 1341 bp, synthetic construct
  misc_feature 1..1341: note = Fusion protein
  source 1..1341: mol_type = other DNA
  CDS 1..1338
atggagtgcagctgcgtgatgctgttcctgctgtccggaaccgcaggcgtgctgtctaggacagaggacctgccaaaggccgtggtgtttctggagccccagtggtaccgcgtgctggagaaggactccgtgacactgaagtgccagggcgcctatagccctgaggataactccacccagtggttccacaatgagagcctgatcagctcccaggcctctagctactttatcgacgcagcaaccgtggacgattccggagagtatcggtgccagaccaacctgagcacactgtccgatccagtgcagctggaggtgcacatcggatggctgctgctgcaggcacctagatgggtgttcaaggaggaggaccccatccacctgcgctgtcacagctggaagaataccgccctgcacaaggtgacatacctgcagaacggcaagggccggaagtacttccaccacaattctgacttttatatccccaaggccacactgaaggatagcggctcctatttttgcagaggcctggtgggcagcaagaacgtgtcctctgagaccgtgaatatcaccatcacacagggactggcacaggtgcagctgcagcagagcggagcagagctggcaaggcctggagcctccgtgaagatgtcttgtaaggccagcggctacaccttcacacggtatacaatgcactgggtgaagcagagaccaggacagggactggagtggatcggatacatcaacccttcccgcggctacaccaactataatcagaagtttaaggacaaggccaccctgaccacagataagagctcctctacagcctacatgcagctgagctccctgacctctgaggacagcgccgtgtactattgcgccagatactatgacgatcactactgtctggattattggggccagggcaccacactgacagtgtctagcgtggagggaggctccggaggctctggaggcagcggcggctccggaggagtggaccagatcgtgctgacccagtccccagcaatcatgtctgccagccctggagagaaggtgaccatgacatgctctgcctcctctagcgtgagctacatgaattggtatcagcagaagtctggcacaagcccaaagcggtggatctacgacacctccaagctggcatctggagtgccagcacacttcagaggctctggcagcggcacctcctattctctgacaatctccggaatggaggcagaggatgcagcaacctactattgtcagcagtggtcctctaaccccttcacctttggctctggcacaaagctggagatcaatagacatcaccaccaccaccactga

SEQ ID NO: 8 - protein, 446 aa, synthetic construct
  REGION 1..446: note = Synthetic Construct
MECSCVMLFLLSGTAGVLSRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRHHHHHH